(19) United States
(12) Patent Application Publication
(10) Pub. No.: US 2014/0113362 A1
(43) Pub. Date: Apr. 24, 2014

(54) Closed Algae Cultivation System

(71) Applicant: INPOWER BIOENERGY TECHNOLOGY CORP., New Taipei City (TW)

(72) Inventors: Ming-luen HUANG, New Taipei City (TW); Mong-yu CHANG, New Taipei City (TW)

(73) Assignee: INPOWER BIOENERGY TECHNOLOGY CORP., New Taipei City (TW)

(21) Appl. No.: 13/854,030

(22) Filed: Mar. 29, 2013

(30) Foreign Application Priority Data
Oct. 19, 2012 (TW) 101219487

(51) Int. Cl. C12M 1/00

(52) U.S. Cl. CPC C12M 21/02
USPC 435/292.1

(57) ABSTRACT

Disclosed herein is an improved closed algae cultivation system that has an algae supplier, a first conduit, at least two containers, and at least two light light-emitting devices. The algae supplier is coupled to the at least two containers via the first conduit. Each container includes at least one sidewall, a bottom and an upper lid, which together define an accommodating volume for accommodating an algal stock culture from the algae supplier. Each container is made of at least one opaque material and includes a first opening disposed on one of the at least one sidewall, and a second opening disposed at the bottom. The at least two light-emitting devices are respectively coupled to the upper lid of each container and extend therefrom along the longitudinal direction to the container such that at least a portion of the light light-emitting devices is submerged in the liquid algal stock culture

### CROSS-REFERENCE TO RELATED APPLICATION

This application claims priority to Taiwan application no. 101219487, filed Oct. 19, 2012, the entireties of which is incorporated herein by reference.

### BACKGROUND OF THE INVENTION

1. Field of the Invention

The present disclosure relates to a closed algae cultivation system. More particularly, the present disclosure relates to a light-impermissible closed algae cultivation system that cuts off external lights.

2. Description of Related Art

Microalgae are unicellular protists typically found in freshwater and marine systems. Microalgae have high economic values; for example, lipid-rich microalgae such as Schizochytrium sp. are used for the manufacture of biofuels, whereas microalgae with high protein content (e.g., Tetraselmis sp.) or high polysaccharide content (e.g., Porphyridium sp.) are used in foods, feeds, nutritional supplements or pharmaceuticals.

Common cultivation methods of algae include open cultivation systems and closed cultivation systems. In open cultivation systems, algae are cultivated in open ponds having large areas in which sunlight is used as the primary light source for the photosynthesis of algae. However, the open cultivation systems need large land areas for considerable mass yields, and the water levels are affected from evaporation and rainfall. Biomass productivity is also limited by contamination with unwanted algal species, organisms that feed on algae or other poisonous particles. Closed cultivation systems, also known as photo-bioreactor (PBR) or bioreactor, use closed containers made of transparent materials for optimized light exposure. Such closed-system design overcomes the contamination and evaporation problems encountered in open systems. The most widely used PBR is a tubular design, which has a number of clear transparent tubes or barrels with a large surface area-to-volume ratio to optimize light penetration from the environment. However, for commercialized cultivation systems that require considerable mass yields, there are unavoidable blind spots where the light could not reach. Therefore, some light-guiding devices are devised so as to introduce the external light into the container. Yet, light-guiding structures have limited transmission range, and the light intensity decreases as the distance from the light source decreases, thereby resulting in uneven light distribution within the container. The blind spot and uneven light distribution would jeopardize the mass yield of the algae cultivation system.

In view of the foregoing, there exists a need in the art for providing a novel closed algae cultivation system to improve the mass yield thereof.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to an improved closed algae cultivation system which is made of light-impermissible material for cutting off external lights so as to precisely control the wavelength, color temperature, frequency, and irradiation time of the light within the container(s) of the system. Therefore, the growth rate of the cultivated algae could be improved.

To this purpose, according to one embodiment of the present disclosure, the closed algae cultivation system comprises an algae supplier, a first conduit, at least two containers, and at least two light-emitting devices. The algae supplier is configured to supply the culture stock required for algae cultivation, which includes the algae, a liquid culture medium (e.g., bold modified basal freshwater nutrient solution) and/or other substances (such as, minerals, trace elements, vitamins, salts, CO₂-containing water, etc.). Structurally, the algae supplier is in fluid communication with each of the containers via the first conduit so as to supply the algal culture stock into each of the container. Each container comprises at least one sidewall, a bottom and an upper lid, which together define an accommodating volume for accommodating the algal culture stock. Each container is made of light-impermissible (or opaque) material(s). The container has a first opening disposed on one of the at least one sidewall and coupled to the first conduit therethrough. The container also has a second opening disposed on the bottom and configured for draining the matured algae. The light-emitting devices are respectively coupled to the upper lid of each container. Each light-emitting device extends downwardly from the upper lid along the longitudinal direction of the container such that at least a portion of the light-emitting device is configured to be submerged in the algal culture stock in each container.

According to optional embodiments of the present disclosure, the sidewall has a height H, and the algal inlet is disposed at ⅔·H to 9/10·H, all being measured parallel to the longitudinal axis from the bottom. In preferable optional embodiments, the algal inlet is disposed at ¾·H to ⅘·H.

According to one embodiment of the present disclosure, the closed algae cultivation system further comprises a second conduit coupled to the second opening of each container, so as to establish a fluid communication among the containers.

According to another embodiment of the present disclosure, the closed algae cultivation system further comprises a third conduit which is coupled to the first conduit, the second conduit, and the algae supplier, so as to established a closed-loop fluid path.

The closed algae cultivation system according to the present disclosure may further comprise a harvest tank coupled to the second conduit so as to provide a temporary accommodation space for storing the liquid culture media and algae drained from the containers of the closed algae cultivation system. The harvest tank is also advantageous in that it may improve the production efficiency of the present closed algae cultivation system by shorting the time required for converting to another cultivation batch.

According to one optional embodiment of the present disclosure, a plurality of harvest control units are disposed in the second conduit. Each of these harvest control units is configured to control (e.g., allow or block) the flow of the liquid in the second conduit 122.

In certain other embodiments of the present invention, the closed algae cultivation system has a third opening disposed on the upper lid. The third opening allows the release of oxygen generated during the process of algal cultivation.

Moreover, the present closed algae cultivation system further comprises a forth conduit disposed outside the containers and coupled to the third opening. The forth conduit is configured to collect the oxygen generated during the process of algal cultivation to a gas collector disposed outside the container for further use. To this end, the present closed algae cultivation system may optionally comprise a gas detector disposed on the inner surface of the sidewall or upper lid of the container for detecting the oxygen level within each container.

According to one embodiment of the present disclosure, the closed algae cultivation system further comprises a liquid supplier coupled to the algae supplier and configured to provide a solution to the algae supplier. Moreover, the liquid supplier further comprises a CO₂ compressor configured to provide a gas composition comprising at most 100% (v/v) carbon dioxide, which is used to generate a CO₂-containing aqueous solution. The CO₂-containing aqueous solution provides CO₂ required for algae growth.

According to one embodiment of the present disclosure, the light emitting devices are configured to emit light with a color temperature of at least 5000 K; preferably, at least 5500 K; and more preferably, at least 6000 K. Further, the light emitting devices may emit a flickering light having a flicker frequency of 20-60 times per second; preferably, 30-50 times per second; more preferably, 40 times per second.

According to one optional embodiment, the light-emitting device may be light-emitting diodes, incandescent light sources, laser light sources, fluorescent light sources, mercury vapor light sources or optical fibers, or a combination thereof.

According to one embodiment of the present disclosure, one of the containers may have a plurality of light-emitting devices that are evenly spaced apart from each other. According to another embodiment of the present disclosure, the light-emitting devices are arranged in multiple circular layers so as maximize the irradiation range of the light-emitting devices, provide a more uniform light distribution within the container, and improve the production efficiency of the present closed algae grower.

According to one embodiment of the present disclosure, the closed algae cultivation system further comprises a maturity monitor disposed on the inner surface of the sidewall of at least one container. The maturity monitor is operable to monitor/detect the maturity of the algae cultivated in the container without disrupting the closed system. For example, the maturity monitor may be a chlorophyll meter or a spectrometer.

Alternatively, in some embodiments, the present closed algae cultivation system may further comprise a maturity monitoring tube disposed outside at least one of the containers. The maturity monitoring tube has two ends respectively coupled to the sidewall, and thereby it forms a fluid communication with the interior of the container such that the liquid within the container is allowed to flow toward the maturity monitoring tube via the inlet at the lower end of the tube. Also, when the surface level of the liquid within the container is higher than the outlet at the upper end of the tube, the liquid within the maturity monitoring tube flows back into the container. The maturity monitoring tube is made of a transparent material so as to allow the detection of the algae maturity in the container without disrupting the closed system from the exterior.

According to one embodiment of the present disclosure, the present closed algae cultivation system further comprises a reflective layer disposed on the inner surface of the container. The reflective layer could be a mirror or other mirror-like structure. For example, the reflective layer is made of at least one metallic material or other light-reflecting materials. The reflective layer is operable to reflect light emitted from the light-emitting device(s) so as to increase the irradiation range and avoid the dead spot of light. In this way, the algae distributed within the container could receive sufficient light.

According to one optional embodiment, the present closed algae cultivation system further comprises a thermal sensor disposed on the inner surface of the sidewall or upper lid, so as to detect the temperature of the liquid culture medium within the container.

In various optional embodiments, the bottom of the container of the present closed algae cultivation system may be flat or conical.

Further, in some optional embodiments, the present closed algae cultivation system further comprises a sealing member. The sealing member is operable to couple the upper lid with the container so as to seal the closed algae grower.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A is a schematic diagram illustrating the closed algae cultivation system 100 according to one embodiment of the present disclosure;

FIG. 1B is a schematic diagram illustrating the closed algae cultivation system 100′ according to another embodiment of the present disclosure;

FIG. 2 is a schematic diagram illustrating the container 210 according to one embodiment of the present disclosure;

FIG. 3 is a schematic diagram illustrating the container 310 according to another embodiment of the present disclosure;

FIG. 4 is a schematic diagram illustrating the light-emitting device 450 according to one embodiment of the present disclosure;

FIG. 5 is a top view illustrating the arrangements of light-emitting devices 550 according to one embodiment of the present disclosure; and

FIG. 6 is a top view illustrating the arrangements of light-emitting devices 650 according to another embodiment of the present disclosure.

FIG. 7 is a curve diagram illustrating the concentration of chlorophyll during the algae cultivation process according to one working example of the present disclosure; and

FIG. 8 is a curve diagram illustrating the pH value of the liquid culture medium during the algae cultivation process according to the above-mentioned working example of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

One purpose of the present invention is to provide an improved closed algae cultivation system with high cultivation efficiency. In particular, the closed algae grower is light-impermissible. In contrast to conventional closed cultivation systems that use transparent containers to allow the penetration of natural light, the present light-impermissible closed algae cultivation system is advantageous in that the users may take complete control over the intensity, color temperature, wavelength, and/or flicker frequency of the light so as to improve cultivation efficiency of the closed algae cultivation system.

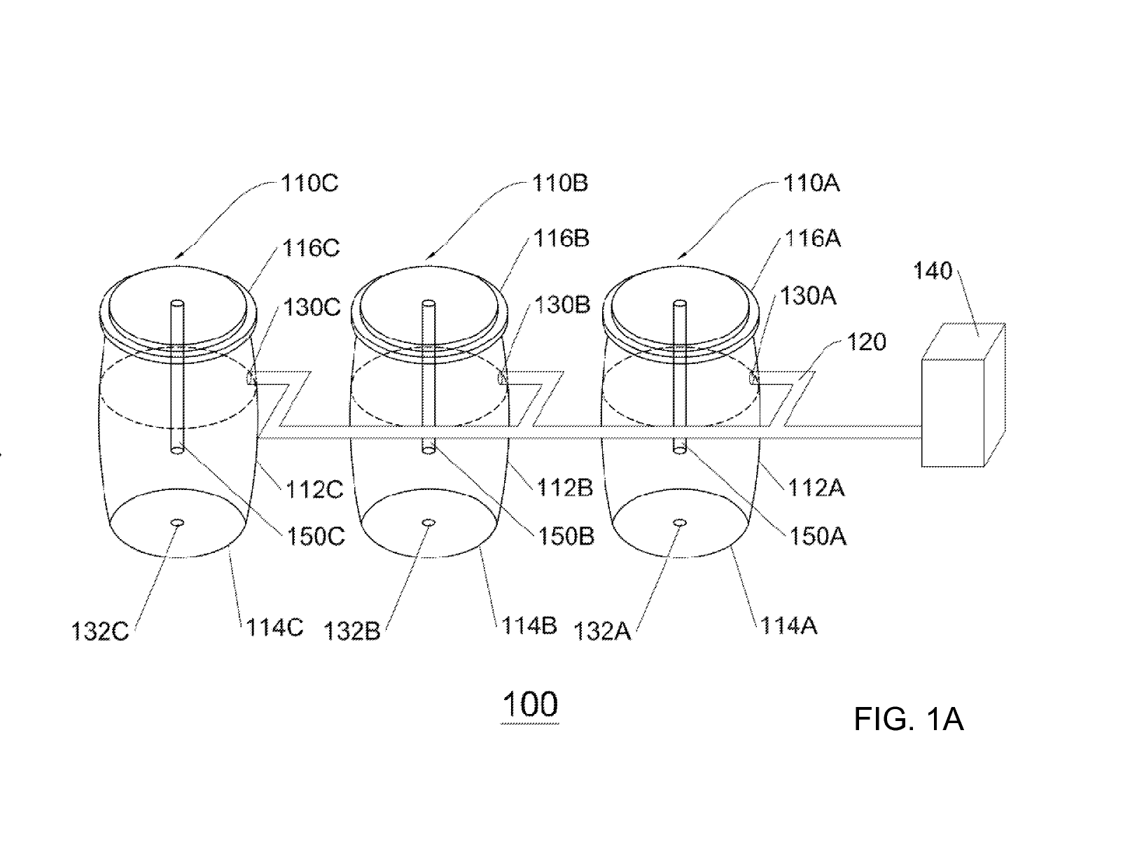

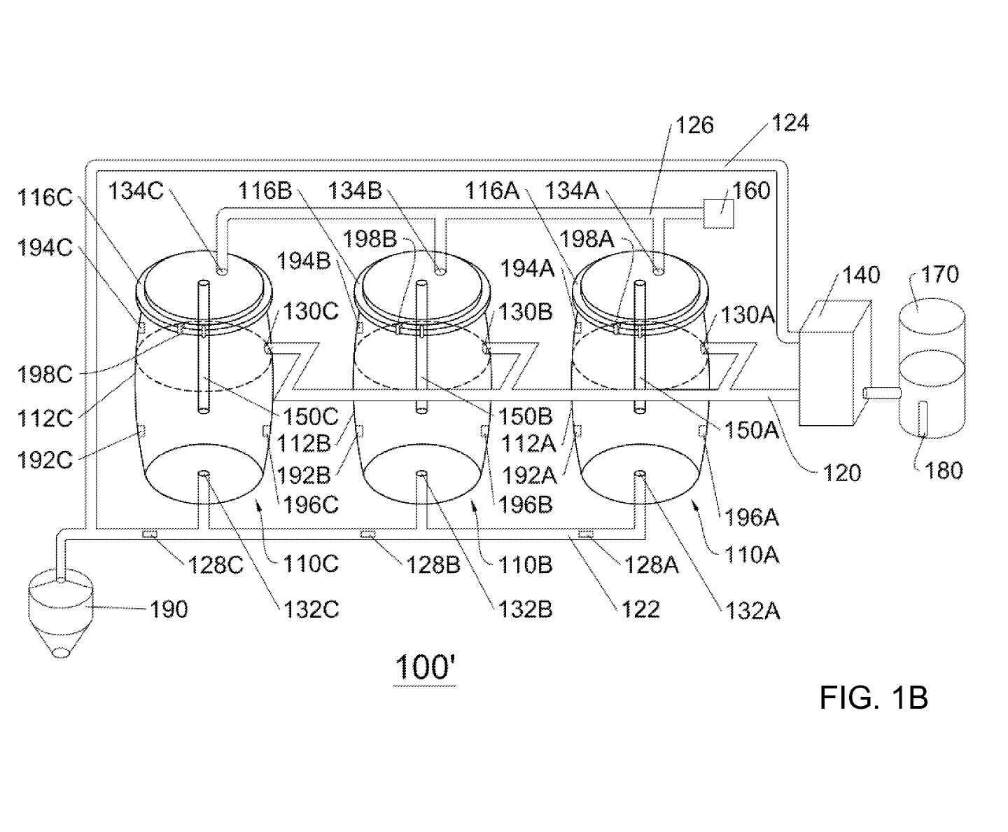

FIG. 1A is a schematic diagram illustrating the closed algae cultivation system 100 according to one embodiment of the present disclosure.

Basically, the present closed algae cultivation system comprises an algae supplier, a conduit, a plurality of containers and a plurality of light-emitting devices.

As an example, rather than a limitation, the closed algae cultivation system 100 comprises three containers (110A, 110B, 110C), and (110A, 110B, 110C) and an algae supplier 140 that are interconnected via a first conduit 120, so as to establish the fluid communication among the containers (110A, 110B, 110C). The algae supplier 140 is configured to supply a culture stock required for algae cultivation. Generally, the algal culture stock includes the algae, a liquid culture medium (e.g., bold modified basal freshwater nutrient solution) and/or other substances (such as, minerals, trace elements, vitamins, salts, CO₂-containing water, etc.). According to various embodiments of the present disclosure, the algal culture stock may include one or more algal species to be cultured.

Specifically, the algae supplier 140 is disposed outside of each container (110A, 110B, 110C), and connected to the first openings (130A, 130B, 130C) of the containers (110A, 110B, 110C) via the first conduit 120, such that the fluid communication among the containers (110A, 110B, 110C) and the algae supplier 140 is established, thereby forming the present closed algae cultivation system 100. Further, during the algae cultivation process, the algae supplier 140 is configured to automatically or semi-automatically adjust the concentration of the liquid culture medium and/or other substances depending on the cultivation parameters, so as to maintain the stability of the cultivation parameters during the cultivation process.

In structure, each of the containers (110A, 110B, 110C) consists of at least one sidewall (112A, 112B, 112C), a bottom (114A, 114B, 114C) and an upper lid (116A, 116B, 116C), which together define an accommodating volume for accommodating the culture stock for algae cultivation. Each container (110A, 110B, 110C) respectively comprises a first opening (130A, 130B, 130C) and a second opening (132A, 132B, 132C). The first openings (130A, 130B, 130C) are respectively disposed on the sidewalls (112A, 112B, 112C), such that the culture stock flows into each container (110A, 110B, 110C) therefrom. The second openings (132A, 132B, 132C) are respectively disposed on the bottoms (114A, 114B, 114C), and configured for draining the cultured algae therefrom.

Each sidewall (112A, 112B, 112C) has a height (H, not shown in the figure) in the longitudinal direction of the container. In various optional embodiments of the present disclosure, the height difference between the first opening (130A, 130B, 130C) and the second opening (132A, 132B, 132C) is about ⅔·H to 9/10·H. Preferably, the height difference is about ¾·H to ⅘·H, and more preferably, about ⅘·H.

During the cultivation, the culture stock would not fill the entire accommodating volume in each container; rather, certain space is saved for accommodating the oxygen generated during the algae growth. Hence, according to various embodiments of the present disclosure, the surface level of the culture stock is lower than, level with, or slightly higher than the first openings (130A, 130B, 130C).

According to principles and spirits of the present disclosure, each container (110A, 110B, 110C) is made of light-impermissible material(s) for completely cutting-off external light source(s). For example, the container (110A, 110B, 110C) may be made of light-impermissible glasses, plastics (polyethylene or high-density polyethylene), or metals (e.g., stainless steel).

At least one light-emitting device (150A, 150B, 150C) is respectively disposed in each of the containers (110A, 110B, 110C). The light emitting device has one end coupled to the upper lid (116A, 116B, 116C) and extends downwardly therefrom such that at least a portion of the light-emitting device (150A, 150B, 150C) is configured to be submerged in the algal culture stock in each container so as to provide sufficient light for algal photosynthesis, which in turns promotes the algal growth rate. In preferred embodiments, each light-emitting device (150A, 150B, 150C) has a length such that the distal end (i.e., the end opposite to the upper lid) of each light-emitting device (150A, 150B, 150C) is near the bottom (114A, 114B, 114C) of the container (110A, 110B, 110C), such that all culture stock in the container (110A, 110B, 110C) would receive sufficient light.

According to one embodiment, the light-emitting device (150A, 150B, 150C) emits a light having a color temperature of at least 5000 K. Preferably, the color temperature is at least 5500 K; and more preferably, at least 6000 K. In one example, the light-emitting device (150A, 150B, 150C) emits a first light having a wavelength of 350-450 nm and a second light having a wavelength of 600-800 nm so as to give a color temperature of about 5500 to 6500 K. Generally, the wavelength composition and color temperature could be adjusted depending on the algal species to be cultivated.

Moreover, in an optional embodiment, the light-emitting device (150A, 150B, 150C) could emit a flickering light to mimic the natural growth environment of algae. For example, the light may have a flicker frequency of about 20-60 timer per second; preferably, about 30-50 times per second; most preferably, about 40 times per second.

Further, the light-emitting device (150A, 150B, 150C) may use any conventional devices or materials as the light source. Examples of suitable light sources include light-emitting diodes, laser light sources, incandescent light sources, fluorescent light sources, mercury vapor light sources, and optical fibers.

In operation, the operator provides the culture stock to the supplier 140, and then the culture stock flows into each container (110A, 110B, 110C) via the first conduit 120. After the algae matures, the algae, together with the liquid culture medium, is drained from each container (110A, 110B, 110C) via the second opening (132A, 132B, 132C).

Although the closed algae cultivation system 100, as illustrated in FIG. 1A, has three container (110A, 110B, 110C), the present invention is not limited thereto. Rather, the closed algae cultivation system may have more containers per users' need to expand the production efficiency of the present system.

FIG. 1B schematically illustrate a closed algae cultivation system 100′ according to another embodiment of the present disclosure. The closed algae cultivation system 100′ of FIG. 1B comprises, in addition to the closed algae cultivation system 100 illustrated in FIG. 1A, a second conduit 122, a third conduit 124, a forth conduit 126, a gas collector 160, a liquid supplier 170, a CO₂ compressor 180 and a harvest tank 190.

Specifically, the second conduit 122 is coupled to each second opening (132A, 132B, 132C) and harvest tank 190, and configured to collect the liquid culture medium and algae drained from each container (110A, 110B, 110C). Further, the third conduit 124 us coupled to the first conduit 120, the second conduit 122 and the algae supplier 140, so as to form a closed-loop fluid path in the closed algae cultivation system 100′, and thereby improve the flowability of the stock culture among the containers (110A, 110B, 110C) and prevent the sedimentation of algae.

In operation, the surface level of the culture stock in the algae supplier 140 is higher than that in each container (110A, 110B, 110C), and therefore, the culture stock would flow from the algae supplier 140 to each container (110A, 110B, 110C) under the action of pressure. When the surface level of the culture stock in the container is level with or slightly higher than the first opening, the culture stock would flow out of the container into the first conduit 120, third conduit 124, and the second conduit 122, in that order. In this way, a reflux path among the containers (110A, 110B, 110C) is established by the first conduit 120, the second conduit 122 and the third conduit 124. This reflux path advantageously avoids the sedimentation of algae, and enhances the growth of the algae. Moreover, if the culture stock is continuously flow from the algae suppler 140 to the containers, the culture stock flowing from the first conduit 120 to the third conduit 124 may further fill the entire third conduit 124, and then flow back to the algae supplier 140. By replenishing the culture stock in the algae supplier 140 with the overflow, a self-contained closed algae cultivation system is achieved.

Moreover, the liquid supplier 170 is coupled to the algae supplier 140 and configured to provide a solution to the algae supplier 140. In one embodiment, the liquid supplier 170 further comprises a CO₂ compressor 180, which provides a gas composition comprising at most 100% (v/v) carbon dioxide. For example, the CO2 concentration in the gas composition is about 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (v/v). In one example, the CO₂ compressor 180 mix carbon dioxide with the air or other gases (e.g., oxygen) at a specific ratio to give the gas composition with the desired CO₂ concentration (e.g., 15% CO2). In another example the gas composition is the atmospheric air that containing about 0.03-0.04% (v/v) CO₂. In yet another example, the gas composition is pure carbon dioxide (i.e., 100% (v/v) CO₂).

Conventionally, bubbles containing carbon dioxide are pumped into water to improve the carbon dioxide content in the water; this process is known as aeration. However, the bubbles in the water would escape into the atmospheric air, thereby resulting in the reduction of the carbon dioxide content. Contrary to the conventional approaches, the present CO₂ compressor 180 exterts a specific pressure such that the gaseous carbon dioxide molecules mix well with the water molecules, and stably distribute in the gap (space) resulted from the molecular bonding between water molecules. This method substantially increased the CO₂ content in the water by 50-100 times, compared to conventional aeration. Also, there no or least air bubbles in the resultant solution; rather, the CO₂ molecules are stably dissolved (inserted) in the liquid molecules, and would not readily escape into the atmospheric air. The liquid supplier 170 then provides the CO₂-containing solution to the algae supplier 140 for providing the CO₂ required during algae growth.

Each of the upper lids (116A, 116B, 116C) has a third opening (134A, 134B, 134C) disposed thereon. The gas collector 160 is disposed outside the containers (110A, 110B, 110C), and connects to each third opening (134A, 134B,134C) via the forth conduit 126. In this way, the oxygen resulted from the photosynthesis during algae cultivation could be discharged from each container (110A, 110B, 110C) via the third opening (134A, 134B,134C), and then guided, by the forth conduit 126, to the gas collector 160 for further use. According to one embodiment of the present disclosure, the system further comprises a plurality of gas detectors (194A, 194B, 194C) configured to detect the oxygen and/or CO₂ concentration in the containers (110A, 110B, 110C). According to various embodiments, the gas detectors (194A, 194B, 194C) may be disposed on the inner surface of the sidewall (112A, 112B, 112C) (such as those illustrated in FIG. 1B), or the inner surface of the upper lids (116A, 116B, 116C).

The temperature control within the cultivation system plays a key role to the algal growth. Therefore, in one optional embodiment, the closed algae cultivation system 100 further comprises a plurality of thermal sensors (196A, 196B, 196C) for detecting the temperature within each container (110A, 110B, 110C). As illustrated in FIG. 1B, each thermal sensor (196A, 196B, 196C) is disposed on the inner surface of the sidewall (112A, 112B, 112C) at a lower position, so as to detect the temperature of the culture stock within the container (110A, 110B, 110C). Alternatively, each thermal sensor (196A, 196B, 196C) may have one end coupled to the upper lid 130, and another end contacting the stock culture.

Also, a plurality of maturity monitors (192A, 192B, 192C) are optionally provided so that the operator could monitor the algae maturity within the container (110A, 110B, 110C) without disrupting the closed system. In one example, the maturity monitor (192A, 192B, 192C) is disposed on the inner surface of the sidewall (112A, 112B, 112C). For example, the maturity monitor (192A, 192B, 192C) may be a chlorophyll meter or a spectrometer. Specifically, the spectrometer (460 nm) or the chlorophyll meter (excitation (Ex): 460 nm; emission (Em): >665 nm) may be used daily to measure the properties of the light passing through the culture stock in the container (110A, 110B, 110C). When the detected properties indicate that the light absorption is greater than 0.5 or the chlorophyll content is greater than 100 μg/L, it is determined that the algae in the container are ready for harvest.

Alternatively, the closed algae cultivation system 100′ optionally comprises a maturity monitoring tube (not shown) disposes outside of the container 110C. The maturity monitoring tube has two ends respectively coupled to the sidewall 112C, thereby forming a fluid communication within the interior of the container 110C. The fluid communication allows the flow of the culture stock within the container 110C toward the maturity monitoring tube. The excess of the culture stock in the maturity tube would flow back into the container 110C; this mechanism promotes the flow of the culture stock within the system. The maturity monitoring tube is made of a transparent material so as to allow the detection of the algae maturity in the container without disrupting the closed system from the exterior.

In another optional embodiment, the closed algae cultivation system 100′ further comprises a plurality of harvest control unit (128A, 128B, 128C) respectively disposed within the second conduit 122 for selectively controlling (enabling and/or blocking) the flow of the culture stock from each container (110A, 110B, 110C) to the second conduit 122. For example, the growth rate of algae in each container may vary from one another. In this case, the harvest control unit (128A, 128B, 128C) may respectively control the flow of the culture stock in each container (110A, 110B, 110C) to the second conduit 122. If only the algae in container 1108 achieve the mature stage, then the harvest control unit 128B allows the drainage of the culture stock in the container 110B. As for other containers (110A, 110C), since the algae contained therein have not matured the harvest control units (110A, 110C) would block the flow of the culture stock from these containers (110A, 110C) to the second conduit 122.

The present closed algae cultivation system 100′ comprises a plurality of optional sealing members (198A, 198B, 198C) that couples the upper lids (116A, 116B, 116C) with the sidewalls (112A, 112B, 112C), so as to seal the gap therebetween.

According to an optional embodiment, each of the containers (110A, 110B, 110C) further comprises a reflective layer (not shown) disposed on the inner surface of thereof. The reflective layer is operable to reflect light emitted from the light-emitting devices so as to increase the irradiation range and avoid the dead spot of light. In this way, the algae distributed within the containers (110A, 110B, 110C) could receive sufficient light. For example, the reflective layer may be a mirror or a mirror-like structure, and could be made of at least one metallic material or reflective material.

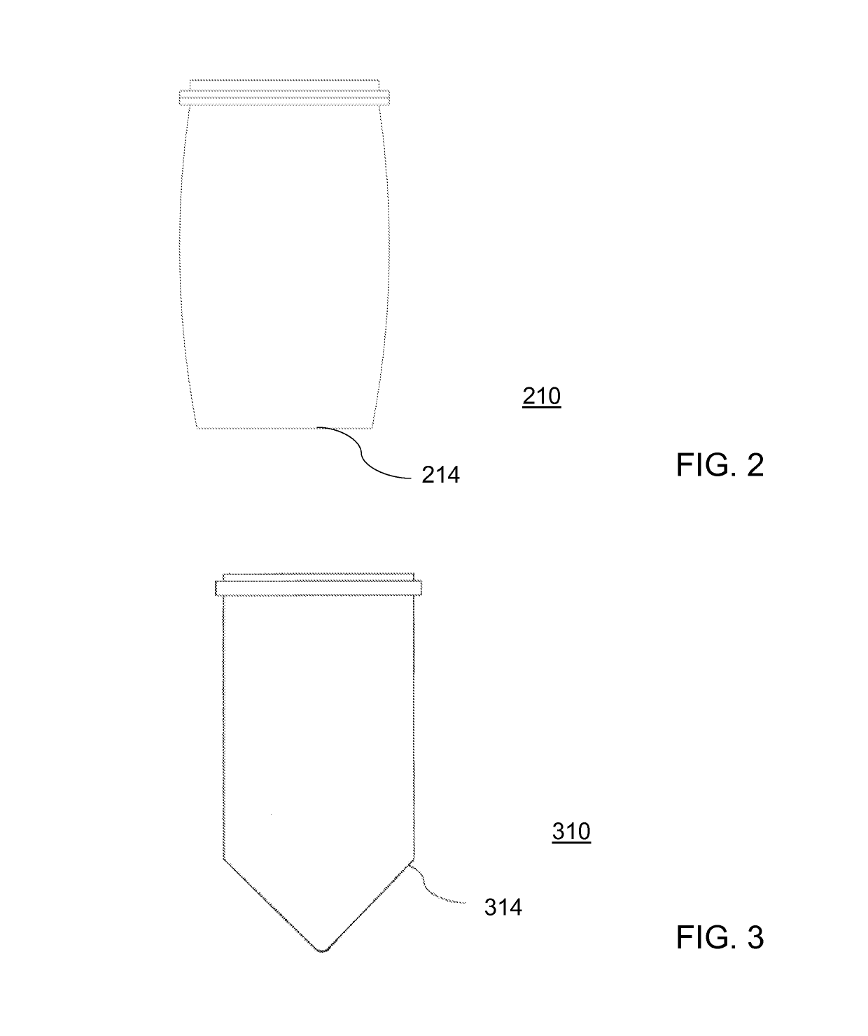

The container may have many different shapes. For example, FIG. 2 is a schematic diagram illustrating a container 210 according to one embodiment of the present disclosure. As illustrated, the container 210 has a substantially circular shape framed by a sidewall. It is also noted that the container 210 is barrel-like in shape which is wider across the center. However, the present disclosure is not limited to this particular shape and a cylindrical container also fall within the scope of the present disclosure. Also, the bottom 214 is flat.

Another example, as illustrated is FIG. 3 is a container 310 according to another embodiment hereof. The container 310 has a substantially rectangular shape framed by sidewalls. Also, the bottom 314 is conical in shape.

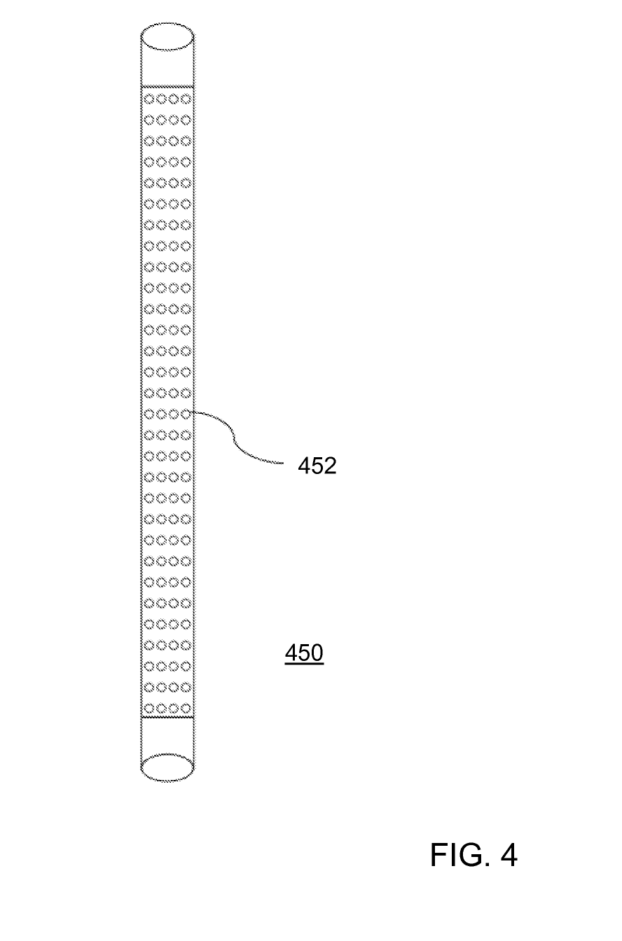

Schematically illustrated in FIG. 4 is a light-emitting device 450 according to one embodiment of the present disclosure. The light-emitting device 450 is cylindrical in shape. In one embodiment, the light-emitting device 450 comprises multiple light-emitting units 452. The light-emitting units 452 are arranged into rows. In certain optional embodiments, the light-emitting device 450 emits light with a flicker frequency of 20-60 times per second, so as to mimic the natural growth environment. In one embodiment, all the light-emitting units 452 are on or off at the same time. In another embodiment, the light-emitting units 452 are on and off alternately.

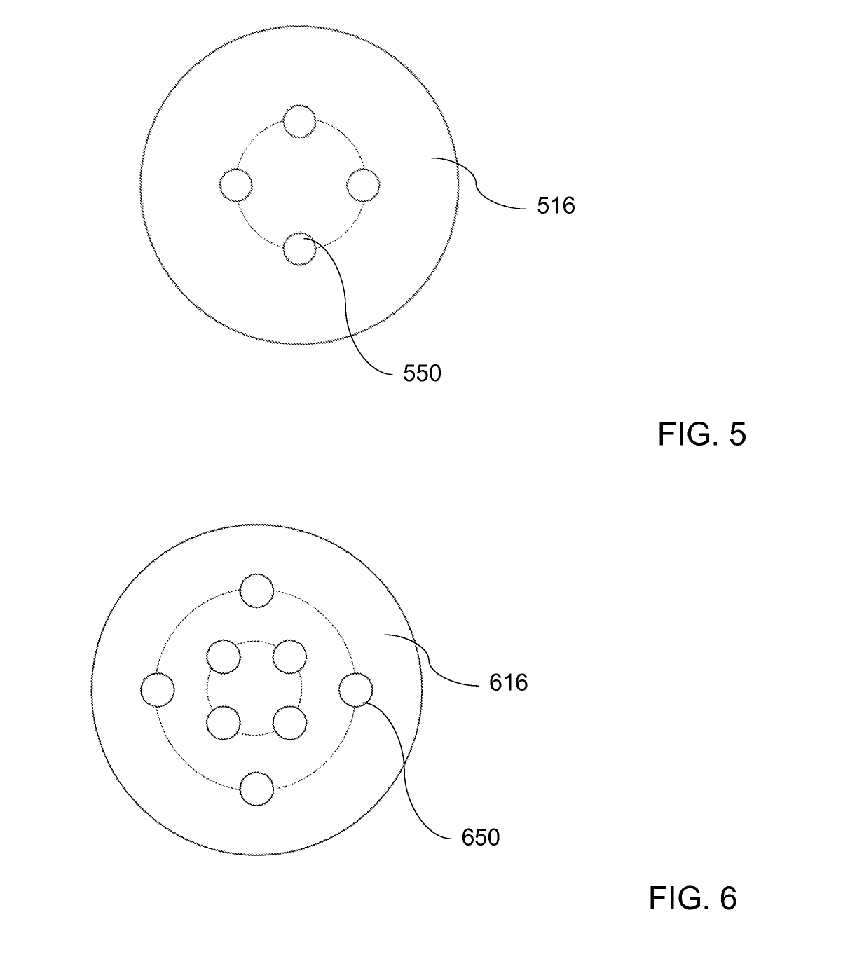

FIG. 5 is a top view illustrating the arrangements of light-emitting devices 550 according to one embodiment of the present disclosure. In this example, the upper lid 516 has four light-emitting devices 550 coupled thereto, and these light-emitting devices 550 are evenly spaced apart from each other so as to maximize the irradiation range of the light-emitting devices 550 and to provide a more uniform light distribution within the container (not shown). This arrangement improves the production efficiency of the present closed algae grower.

FIG. 6 is a top view illustrating the arrangements of light-emitting devices 650 according to one embodiment of the present disclosure. In this example, the upper lid 616 has eight light-emitting devices 650 coupled thereto, and these light-emitting devices 650 are arranged in multiple circular layers. Specifically, four of the eight light-emitting devices 650 are arranged in an inner circle, while the remaining four light-emitting devices 650 are arranged in an outer circle. This arrangement is designed to maximize the irradiation range of the light-emitting devices 650 and to provide a more uniform light distribution within the container (not shown), and therefore, it improves the production efficiency of the present closed algae grower.

### EXAMPLES

In the following working examples, the present closed algae cultivation system was used to cultivate algae using different light sources (high-intensity LED, weak-intensity LED, and fluorescent lamp). The algal culture stock contained two algal species mixed in a 1:1 ratio, Chorella minutissima (3 to 5 μm) and Isochrysis galbana (5-6 um×2-4 um×2.5-3 um). The respective light composition in each group are summarized in Table 1.

TABLE 1

| | Wavelength | Color Temp. | Wattage | Luminous flux |
|---|---|---|---|---|
| | (nm) | (K) | (watts) | (lumen/watt) |
| High-intensity | 430-680 | 6100 | 63 | 110 |
| LED | | | | |
| Weak-intensity | 470-640 | 5700 | 48 | 110 |
| LED | | | | |
| Fluorescent lamp | 500-680 | 4500 | 28 | 63 |

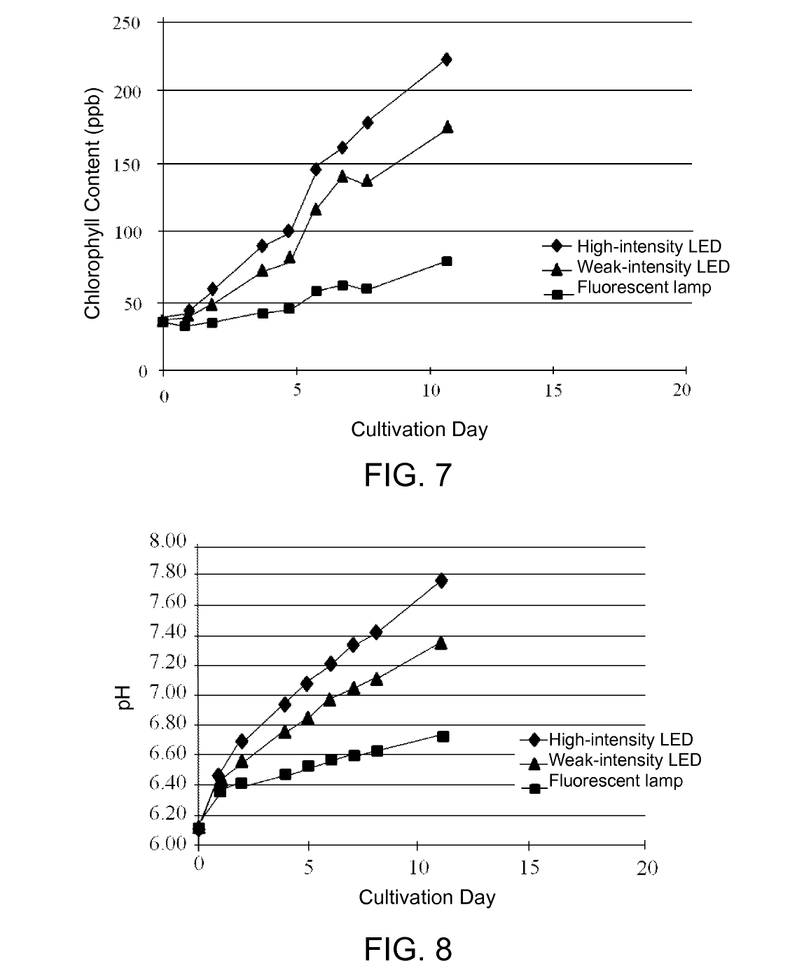

The experimentation condition was as follows. The algae were cultivated in an 100 L container. The culture medium was Bold Modified Basal Freshwater Nutrient Solution and the liquid culture media comprised about 6.8 wt % CO₂. The light was given continuously 24/7. The results are summarized in FIG. 7 and FIG. 8.

FIG. 7 illustrate the chlorophyll content of the container during the cultivation process. As seen in FIG. 7, the group treated by the high-intensity LED topped the others in terms of the increase of the chlorophyll content. At day 11, the content of chlorophyll A (Chl A) reached 225 ppb; in comparison, the Chl A content in the group treated by the fluorescent lamp was only 75 ppb. These data indicate that the high-intensity LED group has the highest growth rate.

The change of the pH value of the culture stock during the cultivation process is summarized in FIG. 8. As illustrated, at day 11, the pH value of the high-intensity LED group was about 7.79, indicating a rapid algal growth. In contrast, the growth rate of the fluorescent lamp group was worst. The respective average growth rates for the three groups were high-intensity LED group: 0.281; low-intensity LED group: 0.249, and fluorescent lamp group: 0.115. The growth rate of the high-intensity LED group is more than twice that of the fluorescent lamp group. Therefore, the time required for cultivating algae are greatly reduced.

The above data suggest that a better production efficiency may be achieved by the complete control of the wavelength, color temperature and frequency light according to embodiments of the present disclosure.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A closed algae cultivation system, comprising:
an algae supplier, configured to provide a culture stock for algae cultivation;
at least two containers, each container is made of at least one light-impermissible material, and comprises:
a bottom, at least one sidewall and an upper lid, which together define an accommodating space,
a first opening, disposed on the sidewall, and
a second opening, disposed on the bottom, wherein the height difference between the first opening and the second opening is about ⅔ to 9/10 of the height of the sidewall;
a first conduit, coupled to the algae supplier and the first opening of each of the containers so as to form a fluid communication thereamong and allow the flow of the culture stock into the containers; and
at least two light-emitting devices, respectively coupled to the upper lid of the containers and extend downwardly such that at least portion of each light-emitting device is configured to be submerged in the liquid stock of each container.

2. The closed algae cultivation system of claim 1, further comprising a second conduit coupled to the second opening of each container and configured to establish a fluid communication between the at least two containers.

3. The closed algae cultivation system of claim 2, further comprising a third conduit connected to the first conduit, the second conduit and the algae supplier, and configured to established a closed-loop liquid path in the closed algae cultivation system.

4. The closed algae cultivation system of claim 2, further comprising a harvest tank coupled to the second conduit, and configured to collect the stock culture drained from each container.

5. The closed algae cultivation system of claim 1, wherein the upper lid further comprises a third opening disposed thereon for discharging gas.

6. The closed algae cultivation system of claim 5, further comprising a forth conduit coupled to the third opening of each container to establish a fluid communication between the at least two containers.

7. The closed algae cultivation system of claim 6, further comprising a gas collector coupled to the forth conduit.

8. The closed algae cultivation system of claim 1, further comprising a liquid supplier coupled to the algae supplier, wherein the liquid supplier comprises a CO₂ compressor configured to mix a gas composition comprising CO₂ with a liquid to produce a CO₂-containing solution.

9. The closed algae cultivation system of claim 8, wherein the gas composition has a CO₂ concentration in a range of >0% and 100% (v/v).

10. The closed algae cultivation system of claim 8, wherein the CO₂-containing solution has a CO₂ concentration of at least 3 wt %.

11. The closed algae cultivation system of claim 1, further comprising a reflective layer disposed on the inner surface of each container.

12. The closed algae cultivation system of claim 1, wherein each of the at light-emitting devices is selected from the group consisting of, light-emitting diodes, laser light sources, incandescent light sources, fluorescent light sources, mercury vapor light sources, and optical fibers.

13. The closed algae cultivation system of claim 1, wherein the at least two light-emitting devices emit a light having a color temperature of at least 5000 K.

14. The closed algae cultivation system of claim 1, wherein the at least two light-emitting devices emit a light with a flicker frequency of 20-60 times per second.
